(19) United States
(12) Patent Application Publication
(10) Pub. No.: US 2021/0299166 A1
(43) Pub. Date: Sep. 30, 2021

(54) COMPOSITIONS FOR MAINTAINING ELECTROLYTE BALANCE AND URINARY TRACT HEALTH

(71) Applicant: ALLGOOD BEVERAGE COMPANY, Folsom, LA (US)

(72) Inventor: Brian Lee BROTHERS, Folsom, LA (US)

(21) Appl. No.: 17/212,408

(22) Filed: Mar. 25, 2021

Related U.S. Application Data
(60) Provisional application No. 62/994,399, filed on Mar. 25, 2020.

(51) Int. Cl. A61K 33/14; A61P 3/12; A61K 33/10; A61K 33/08; A61K 31/7004; A61P 3/14; A61K 9/48

(52) U.S. Cl. CPC A61K 33/14; A61P 3/12; A61K 33/10; A61K 9/4816; A61K 31/7004; A61P 3/14; A61K 33/08

(57) ABSTRACT

An electrolyte supplement/replacement, in particular a gel capsule, comprises an electrolyte formulation for facilitating sufficient electrolyte replenishment and compounds for maintaining urinary tract health in dehydrated persons, including but not limited to persons who lose substantial amounts of electrolytes due to illness or those who engage in strenuous physical activities in hot climates and/or work environments, such as military personnel, construction workers, sports training, athletes and the like, to improve electrolyte balance, extend endurance, prevent muscle cramps and other effects of dehydration. The supplement comprises sufficient types and amounts of electrolytes to facilitate rapid return to electrolyte homeostasis in a dehydrated person.

### FIELD OF THE INVENTION

The invention is directed generally to the field of electrolyte replenishment, in particular an electrolyte supplement in gel capsule form comprising an electrolyte formulation for replenishment and compounds for maintaining urinary tract health in dehydrated persons. The invention is also directed to methods of improving electrolyte balance and preventing the dehydrating effects of exposure associated with illness or high temperature environments.

### BACKGROUND OF THE INVENTION

Dehydration is a common complication associated with athletic training and competition and situations requiring strenuous physical effort, such military exercises or construction work. Strenuous athletic or work activity causes sweating and can be a risk for dehydration and concomitant water and electrolyte loss through sweating. When coupled with hot climates or environments, dehydration risk increases and can present significant problems for the individual.

Dehydration conditions range from mild to severe. While dehydration is most commonly associated with water loss, in almost all instances water loss is couple with glucose and electrolyte loss. In mild dehydration, subjects experience mild effects, such as lethargy and sleepiness, dry skin, mouth and lips, constipation, dizziness, headache, and thirst. In more severe dehydration, sweating may cease, heart rate and breathing increase, fever may occur, chills, dark urine, low blood pressure, and dark eyes can also occur. In extreme circumstances, heat stroke may occur or death.

Severe illnesses, such as cholera, dysentery, and Ebola, among other illnesses, can also cause subjects to lose vast volumes of body water and electrolytes, leading to severe dehydration. This loss contributes to the high rate of mortality associated with these diseases.

Dehydration affects electrolyte balance. Sodium, potassium and calcium levels may be increased when body water volume is lost. Sodium is an electrolyte that regulates the amount of water available to cells. When body water volume is lost, sodium levels may increase resulting in hypernatremia, which may result in loss of energy, confusion, seizures, or coma, among other complications, and must be resolved. Potassium levels may also increase affecting cardiovascular function. Calcium levels may also increase due to low body water volume, resulting in kidney or cardiovascular complications. It is important to note that these “hyper” or increased levels of electrolytes are cause by a loss of body water and are not a true indication of actual serum electrolyte levels. When body water is fully replenished, there is a chance that electrolyte levels will remain low, with the effects of dehydration still being seen, if electrolytes are not also replenished.

One of the first indicators of dehydration is thirst, and the natural tendency is to replace water. There are several ways to address loss of water, whether due to disease or exertion. First, thirst is often addressed by providing copious amounts of water. While water is much needed, it is possible to drink too much water. If only water is replenished, water intoxication can result. Previous “hyper” or increased levels of electrolytes are diluted with water replenishment thus resulting in “hypo” or lower levels of electrolytes as water levels return to normal. Failure to replace electrolytes lost along with the water can compound dehydration issues and lead to organ damage if not resolved.

As an alternative, in the field, intravenous fluids may be used to replenish both water and electrolyte loss; however, their use is not always practical and may be expensive. Moreover, it is not always possible to assess electrolyte levels in the field to determine whether adequate replenishment has occurred.

Salt tablets are also popular in hot environments and are sometimes administered prior to exposure to facilitate the body's ability to acclimate to hot environments; however, acclimation requires several days. It is also possible to overconsume salt tablets resulting in an impairment of acclimation rather than facilitation of it. Moreover, supplementation of sodium to the exclusion of other essential electrolytes may also be problematic. It may also facilitate loss of other electrolytes as the kidney adapts to excreting excess sodium.

There are other effects of dehydration that are encountered in the field. Dehydration can lead to concentrated urine, which in turn can irritate the bladder. While adequate water consumption in hot environments helps the body to flush out bacteria that may cause a urinary tract infection (UTI), in the field, adequate water consumption may not be possible. However, there may not be facilities for use and athletes, workers, military personnel and others may tend to hold urine, putting themselves as risk for bacterial infection. This may be particularly true for female workers where hormonal fluctuations can affect normal flora.

In the past decades, considerable research and work relating to electrolyte and carbohydrate supplementation has been ongoing, particularly in the area of exercise physiology. The use of electrolyte replacement supplements to satisfy various requirements of athletes and other persons while training or engaging in strenuous exercise is known. Electrolyte supplements are also available to counteract the effects of diseases that dehydrate, particularly in children. There are many electrolyte and carbohydrate supplements on the market which claim to replenish carbohydrates, essential electrolytes and water lost from the body during physical exercise. Some supplements provide only carbohydrates (sugars), and others provide a level of electrolytes, which may not be enough to replenish what is actually lost. Electrolyte and carbohydrate supplements are available in liquid, powder and tablet form.

Examples of electrolyte and carbohydrate supplement liquid compositions are known in the art. By way of example only, U.S. Pat. No. 4,322,407 discloses an electrolyte drink comprising electrolytes, citrates, sucrose, dextrose, ascorbic acid and pyridoxine to be mixed with water. U.S. Pat. No. 7,740,893 discloses a sports drink stated to be useful for enhancing glucose uptake into the muscle and extending endurance during physical exercise. The drink contains a mixture of carbohydrates, one or more proteins, wherein the carbohydrate to protein ratio is in the range of 2.8 to 4.2 parts of carbohydrate to 1 part of protein. U.S. Pat. No. 10,449,165 is directed to methods, compositions, systems and kits that deliver a laxative to patients along with electrolyte replenishment to counteract electrolyte loss caused by diarrhea associated with the use of laxatives. U.S. Patent Publication No. 2006/0068005 is directed to a chewable electrolyte tablet comprising at least one nutritional compound, such as a vitamin, a non-carbohydrate sweetening agent to improve taste and a gastrointestinal (GI) soothing agent to prevent GI upset from electrolyte concentrations. U.S. Publication No. 2017/0056441 is directed to a nutritional supplement comprising a mixture of Vitamin D3, one or more potassium compounds, creatine, and magnesium glycinate.

Use of electrolyte supplements is not without disadvantages. While there are many products on the market, few, if any, provide a complete replacement of all essential electrolytes lost due to water loss or they may not include enough of the right type of electrolytes to facilitate full replenishment. In addition, many electrolyte supplements are not palatable and contain carbohydrates, which may not always be necessary or required, although they may improve taste. Palatability is a concern for products that have high salt content. High salt or electrolyte content may cause GI upset as well.

Some products, such as tablets or even powders, are not readily bioavailable due to the type of components, salt forms and other additives used in the product. Salt tablets, discussed above, replenish sodium, but take time to act and do nothing to replenish other vital electrolytes lost. In some instances, they may not be completely absorbed.

Electrolyte-containing powders are also available. While seemingly addressing a need, these supplements require mixing with water, which may not be practical or feasible in the field. Some supplements may be provided in a form such as in liquid packets or bottles that require a user to carry additional liquids. While products in powder (when mixed) or liquid form generally are better absorbed, in many situations, water for mixing is not readily available in the field or liquid packets or bottles may leak or be damaged, resulting in a loss of liquid.

None of the known available electrolyte supplement or sports drinks may be used as the exclusive source of electrolyte support during periods of athletic training or competition or situations where significant physical energy is being expended such as during military exercises or construction work. In short, none of the available electrolyte supplement drinks will provide for the complete electrolyte requirements of a body under stress, particularly in hot environments.

A need remains for an easy to use electrolyte supplement for use in severe illness and or electrolyte loss incurred by strenuous physical activity, particularly in hot environments. In addition, there is a need to provide an electrolyte supplement that comprises electrolytes of the type and in an amount sufficient to facilitate complete replenishment. Ideally, the electrolyte supplement will not require mixing or other preparation in the field and may be supplied prior to engaging in activity. It will also not require carrying liquid packets or bottles, which may evaporate or leak. In addition, in many fields, there is a demand to preserve water resources, such that an electrolyte supplement that does not require mixing with water is a significant need.

Accordingly, it would be beneficial to provide an electrolyte supplement that avoids the disadvantages associated with traditional electrolyte supplements/replacements. Practically, the electrolyte supplement would be able to be taken without water or any other consumable liquid. It would also be beneficial to provide an electrolyte supplement having reduced levels of carbohydrates and one in a form that does not irritate the gastrointestinal system of the user.

An ideal electrolyte replacement formulation would be one that is useful for those persons who lose substantial amounts of electrolytes due to illness as well as those who engage in strenuous physical activities in hot climates and/or work environments, such as military personnel, construction workers, sports training, athletes and the like. Such as formulation would also improve electrolyte balance, improve and extend performance and endurance, and prevent muscle cramps and muscular damage caused by electrolyte loss.

A preferred electrolyte supplement will comprise all essential electrolytes implicated in dehydration states and anti-infectives that reduce or eliminate urinary tract infections associated with dehydrated states. The electrolyte supplement will also be bioavailable within a pharmaceutically acceptable time after ingestion.

An important aspect of electrolyte supplementation is balance. It is well known that regulation of electrolytes plays an important role in a large number of cellular and bodily functions. Many electrolytes work in concert to facilitate movement of other electrolytes across the cell walls. As one example, in the paracellular pathway, sodium and potassium transports are generally increased by magnesium. Magnesium works by a number of different mechanisms as a regulator of the sodium/potassium pump. As one mechanism, magnesium stimulates sodium/potassium exchange at low concentrations and inhibits it at higher concentrations, which in turn reduces the rate of turnover of the pump. Magnesium also plays a role in the function of the calcium pump and sodium/calcium exchange and can restore electrolyte homeostasis rapidly when provided in amounts sufficient to facilitate the exchange.

Studies have shown that magnesium is a cofactor in transmembrane movements and has profound effects on both solute and water transport in various cells. Magnesium deficiency is followed by potassium deficiency which cannot be equalized by administering potassium alone. Magnesium works with adenosine triphosphate (ATP) to provide the body with energy. ATP cannot work without magnesium. As such, it is an important electrolyte for supplementation and replenishment in dehydrated subjects.

An electrolyte supplement has been developed that meets the aforenoted criteria for ideal/preferred electrolyte supplementation. The electrolyte supplement is in the form of a gel cap, thus not requiring field preparation, comprises essential electrolytes associated with dehydration states in amounts sufficient to prevent or reduce electrolyte loss, is well absorbed and bioavailable and may be given prior to engaging in strenuous activity in hot climates.

The gel cap electrolyte supplement of the invention is a significant improvement over that known in the art. The formulation provides adequate amounts and types of electrolytes and is much more cost effective than other formulations, as less is required to accomplish electrolyte replenishment when compared to other formulations that have similar but lower content of electrolytes. The gel cap avoids gastrointestinal upset associated with high salt concentrations of tablets but allows for ready absorption of electrolytes. The formulation has been tested for disintegration, which occurs in about fifteen minutes, making it comparable to the bioavailability of liquids and/or some powders dissolved in liquids. The inventive formulation provides an added advantage in that it also includes an anti-infective agent.

It is an object of the invention to provide an electrolyte supplement for use by athletes and those engaging in strenuous physical exercise to prevent or reduce the risks associated with electrolyte loss.

A further object of the invention is to provide an electrolyte supplement for use by persons exposed to hot training or work climates or environments, which may be administered prior to engaging in strenuous activity.

Another object of the invention is to provide an electrolyte supplement in a form that allows for ready absorption of electrolytes with little gastrointestinal upset.

Yet another object of the invention is to provide an electrolyte supplement that is easy to transport and carry in the field and requires no water for mixing to administer.

Still another object of the invention is to provide an electrolyte supplement comprising electrolytes of the types and in the amounts required to facilitate full replacement of electrolytes required for bodily functions.

An important object of the invention is to provide an electrolyte supplement comprising sufficient magnesium content to facilitate the proper functioning of the Na/K pump, the Ca-pump and Na/Ca pump, all necessary to electrolyte homeostasis.

Other objects of the invention will be known to one skilled in the art based on the disclosure herein.

### SUMMARY OF THE INVENTION

The invention is directed to an electrolyte supplement (replacement) for use in the prevention, reduction and/or treatment of electrolyte loss associated with dehydration, particularly in those persons who undertake strenuous physical activity in hot climates or environments. The electrolyte supplement is provided in gel cap form, which is ready to use in the field. There is no preparation required and no need to carry liquid supplements. The concentration of electrolytes in the formulation is greater than that typically found in known electrolyte supplements and thus reduces the amount required to prevent and/or treat dehydration. The gel cap provides gastrointestinal protection and unexpected bioavailability comparable to that achieved with liquid preparations. An added advantage is the inclusion of an anti-infective to prevent bladder irritation and/or urinary tract infections commonly encountered with dehydration.

In one embodiment, the invention is a gel cap formulation comprising:
a. Sodium salts,
b. Potassium salts,
c. Calcium salts,
d. Magnesium salts, and
e. An anti-infective.

In another embodiment, the invention is a method for preventing, reducing or treating electrolyte loss associated with dehydration states comprising the step of administering the gel cap formulation prior to and while engaging in strenuous physical activity.

Other embodiments of the invention will be known to one skilled in the art based upon the disclosure herein.

### DETAILED DESCRIPTION OF THE INVENTION

The invention is directed to an electrolyte supplement (replacement) for use in the prevention, reduction and/or treatment of electrolyte loss associated with dehydration, particularly in those persons who undertake strenuous physical activity in hot environments. The electrolyte supplement is provided in an easy-to-use gel cap form, requiring no preparation and no need to carry water or other liquid supplements in the field. The electrolyte supplement provides gastrointestinal protection from high salt content but unexpectedly is well absorbed and readily bioavailable to the user. The inventive formulation replenishes electrolytes necessary for bodily functions. An added advantage is that, by fulfilling electrolyte requirements, it improves performance by allowing muscle cells to extend duration time thus reducing muscle fatigue. In some instances, the combination of potassium, calcium and magnesium may help offset any blood pressure increases caused by sodium ingestion.

Because it is in gel cap form, the concentration of electrolytes in the inventive formulation can be and is greater than that typically found in known electrolyte supplements and thus reduces the amount required to prevent, reduce and/or treat electrolyte loss associated with dehydration. The gel cap provides unexpected bioavailability comparable to that achieved with liquid preparations. An added advantage is the inclusion of an anti-infective to prevent bladder irritation and/or urinary tract infections commonly encountered with dehydration.

The following definitions are used herein:

“Electrolyte” shall mean and include salts and minerals that can conduct electrical impulses in the body. Electrolytes control fluid balance in the body and are important for fluid balance, acid-base balance, delivery of oxygen to cells, cell wall integrity, the nervous system, cardiac system, muscle contraction, energy generation, and almost every major biochemical reaction in the body. In short, electrolytes allow the body to function and work. Electrolytes include but are not limited to sodium, potassium, magnesium, calcium and chloride.

“Supplement” or “replacement” with respect to the invention means and includes providing additional electrolytes lost through any situation or condition that leads to water loss and associated dehydration states. “Supplement” and “replacement” with respect to electrolytes are used interchangeably herein.

Electrolyte disorders are not only associated with dehydration but may be implicated in a number of disease and clinical states. Dehydration disturbs electrolyte balance creating an “imbalance”. Lack of electrolyte homeostasis can have serious consequences on a cellular level. If not resolved, cellular activity may be impaired, as well as action and resting membrane potentials. Electric instability in cells can lead to a number of serious consequences.

Electrolyte deficiencies may not always be determined through simple serum evaluations, particularly for potassium and magnesium. Only about 2 percent of total body potassium and about 1 percent of total body magnesium is accessible in the extracellular space where it may be assessed by measuring plasma or serum levels. When dehydration with loss of electrolytes occurs, the body attempts to maintain serum concentrations of electrolytes by transporting electrolytes from intracellular stores to the extracellular space, often at the expense of muscle, cardiac, bone and liver cells. This transport, coupled with concomitant water loss, results in normal serum electrolyte levels or a “hyper” level of electrolytes. As a result, it is often difficult to determine electrolyte deficiencies, particularly for potassium and magnesium. Accordingly, for electrolyte loss due to dehydration, a preferred approach is to prevent or reduce electrolyte loss in the field with electrolyte supplements.

It is known that magnesium is important in the functioning of the Na/K pump, the Ca-pump and Na/Ca exchange. (See Schroll, A., “Importance of magnesium for the electrolyte homeostasis—an overview”, 2002, available online at mgwater.com and Hans-Jurgen, A. et al., “Modulation of the Na, K-ATPase by Magnesium Ions”, Biochemistry 56(7): 1005-1016 (2017), doi:10.1021/acs.biochem.6b01243.Epub 2017 Feb. 9). The Na/K pump maintains cellular potassium concentration by active transport across a gradient. It has been well-known that Mg²⁺ is an essential cofactor for activation of enzymatic ATP hydrolysis by ATPase, without being transported through the cell membrane. (Hans-Jurgen, A., supra). In short, magnesium is responsible for the function of ATPase, the enzyme involved in the Na/K pump. When magnesium is deficient, ATPase demonstrates reduced activity. While not wishing to be bound by theory, regardless of mechanism, magnesium plays an important role in the function of the Na/K pump. Similarly, magnesium deficiency is implicated in the efficient functioning of the calcium pump (Ca-pump) and the sodium/calcium exchange (Na/Ca exchange). In short, magnesium facilitates the transport of potassium and calcium in and out of cells and may aid in the absorption of these critical electrolytes.

Further, magnesium deficiency and potassium deficiency are often concurrent. Magnesium deficiency is often followed by potassium deficiency. And it is known that potassium deficiency, i.e., hypokalemia, can be refractory, and not resolved by administration of potassium alone. Additional magnesium may be needed to restore potassium balance. It is therefore critical to assure that adequate magnesium is provided to facilitate the efficient and rapid return to electrolyte homeostasis.

The inventive electrolyte supplement includes not only sodium and potassium, but also magnesium and calcium, all of which work in concert to restore electrolyte homeostasis, in amounts sufficient to replenish electrolytes in dehydration states. Although various salt forms of each electrolyte exist and may be used, preferably the formulation comprises sodium (as sodium chloride), potassium (as potassium chloride or potassium gluconate), calcium (as calcium carbonate, calcium chloride or calcium gluconate) and magnesium (as magnesium oxide, chloride, sulfate or citrate). Although the use of a gel cap for the inventive electrolyte supplement improves gastrointestinal tolerance, both calcium carbonate and magnesium oxide may also facilitate digestion and/or prevent gastrointestinal upset from high salt content.

Because the inventive electrolyte formulation is included in a gel cap, the amounts of each electrolyte as well as total electrolytes that can be incorporated into one gel cap are limited to an extent, but higher than what is typically found in known electrolyte supplements. As such, the inventive electrolyte formulation may require at least two capsules per serving to assure adequate supplementation. Even so, it is far easier to take two fast acting, readily available capsules to achieve electrolyte balance than to prepare multiple powders or carry large amounts of liquid. By way of example only, 60 capsules (2 capsules per serving) is generally equal to 90 bottles of typical sports drinks. A user can carry a 30-day supply of electrolytes in a small light weight container, as compared to carrying more powder packets and the water required for mixing or large volumes of liquid preparations.

The inventive formulation also comprises anti-infectives known to be useful to reduce or eliminate urinary tract infections (UTI's). Suitable anti-infectives include D-mannose, lauricidin, nattokinase, apolactoferrin or lactoferrin, cranberry extract, polyphenols, probiotics, ascorbic acid and other urinary acidifiers. D-mannose is preferred as it is effective against Escherichia coli, which is believed to be responsible for about 90% or UTI's.

The amount of sodium, calcium, potassium and magnesium contained in the inventive electrolyte supplement can vary depending on capsule size. Preferably, sodium is present in the inventive electrolyte formulation in amounts of at least 200 mg per capsule; calcium is present in at least 100 mg per capsule; potassium is present in at least 100 mg per capsule; and magnesium is present in at least 25 mg per capsule. Ideally, the ratio of potassium to magnesium in any one capsule should be at least 4:1. This ratio approximates normal potassium to magnesium ratios in the extracellular fluid.

A particularly preferred embodiment comprises (per capsule):
Sodium chloride 250 mg/capsule
Calcium carbonate 139 mg/capsule
Potassium chloride 108 mg/capsule
Magnesium oxide 25 mg/capsule
D-mannose 75 mg/capsule

Other amounts may be utilized and are within the scope of the invention. Amounts utilized may be limited by capsule size selected. For the purposes of the invention a minimum size for the gel cap would be a number two capsule. No particular material is required for the capsule, provided that the capsule is capable of dissolving in about fifteen minutes. For purposes of the invention, the types and amounts utilized should be adequate for the purpose of fulfilling electrolyte replenishment. The invention contemplates that adequate supplementation requires two capsules per serving of the inventive electrolyte supplement. The number of servings ingested will depend on the particular duration of physical activity and the environment. Comparisons with other electrolyte supplements discussed herein based upon “per serving”, not “per capsule”, electrolyte content.

The invention is further described by the non-limiting examples set forth below.

### Example 1—Certificate of Analysis for Content and Disintegration

The inventive electrolyte supplement formulation in Table 1 below was evaluated.

TABLE 1

| | Milligrams (mg) per two |
|---|---|
| Material | capsule serving |
| Sodium (as chloride) | 500 mg |
| Potassium (as chloride) | 216 mg |
| Calcium (as calcium carbonate) | 279 mg |
| Magnesium (as magnesium oxide) | 50 mg |
| D-Mannose | 150 mg |

The inventive formulation was incorporated into a hard gelatin capsule. Capsules were analyzed by third party testing agencies and certified for conformance to the above formulation specifications, heavy metal tests, and disintegration time. Notably, conformance to content specification was verified after disintegration thus providing an assessment of the amount of electrolytes actually delivered through the gel cap dosage form once released in the body. Results are shown in Table 2 below.

TABLE 2

| | | | Results per two |
|---|---|---|---|
| Analysis | Specification | Method* | capsule serving (**) |
| Appearance | Off-white/white powder blend. | Visual | Conforms |
| | White cap/white body capsule | | |
| Capsule disintegration test | NMT*** 15 minutes | USP 32 <2040> | Conforms |
| Sodium (as chloride) | NLT**** 500 mg/serving | ICP/MS Assay | 552.05 mg/Serving |
| Potassium (as chloride) | NLT 216 mg/serving | ICP/MS Assay | 213.04 mg/serving |
| Calcium (as carbonate) | NLT 279 mg/serving | ICP/MS Assay | 296.55 mg/serving |
| Magnesium (as oxide | NLT 50 mg/serving | ICP/MS Assay | 56.07 mg/serving |
| D-Mannose | NLT 150 mg/serving | HPLC ALA626A | 167.2 mg/serving |

*Method utilized can be either USP or other accepted compendia methods.
(**) All assay testing methods had a variance of +/−10%.
***NMT = “No more than.”
****NLT + “No less than.”

Heavy metal testing (results not shown) utilizing USP test methods resulted in a “pass” rating for arsenic, cadmium, lead, and mercury. Microbiology testing (results not shown) was negative for P. aeruginosa, S. aureus, E. coli, and Salmonella sp.

Third party testing results showed that the inventive electrolyte replacement formulation does not contain any trace contaminants and that the contents were verified as specified.

The results above show that the inventive electrolyte formulation is capable of delivering sufficient types and amounts of electrolytes when incorporated into a gel capsule. The gel capsule disintegration time better than that obtained for coated and uncoated tablets. As such, it is expected that bioavailability would be comparable to that achieved when using liquid electrolyte supplements. The gel capsule has the advantage of being able to deliver higher amounts of electrolytes as compared to currently available electrolyte supplements, while at the same time reducing or eliminating palatability issues and gastrointestinal upset typically encountered with current supplements.

In addition, the inventive electrolyte formulation includes a sufficient amount of magnesium to assure proper functioning of the Na/K pump, the Ca pump and Na/Ca pump, thus improving the time in which electrolyte homeostasis can be restored.

While in accordance with the patent statutes the best mode and preferred embodiment have been set forth, the scope of the invention is not limited thereto, but rather by the scope of the attached claims.

What is claimed is:

1. An electrolyte supplement consisting of:
a. sodium,
b. calcium
c. potassium,
d. magnesium, and
e. a bladder anti-infective,
wherein the electrolyte supplement is provided in the form of a gel cap, and
wherein the gel cap disintegrates in about fifteen minutes under a standard USP disintegration test.

2. The electrolyte supplement of claim 1, wherein sodium is provided as sodium chloride, wherein calcium is provided as calcium carbonate, wherein potassium is provided as potassium chloride, and wherein magnesium is provided as magnesium oxide.

3. The electrolyte supplement of claim 1, wherein the anti-infective comprises D-mannose, lauricidin, nattokinase, apolactoferrin, lactoferrin, cranberry extract, polyphenols, probiotics, ascorbic acid, or urinary acidifiers.

4. The electrolyte supplement of claim 2, wherein sodium chloride is present in an amount of at least 200 mg per gel capsule, wherein calcium carbonate is present in an amount of at least 100 mg per capsule, wherein potassium chloride is present in an amount of at least 100 mg per capsule, wherein magnesium oxide is present in an amount of at least 25 mg per capsule.

5. The electrolyte supplement of claim 4, wherein the ratio of potassium to magnesium is at least 4:1.

6. A gel capsule comprising an electrolyte supplement for preventing, reducing or treating dehydration, wherein the electrolyte supplement consists of:
a. sodium chloride present at 250 mg/gel capsule,
b. calcium carbonate present at 139 mg/gel capsule,
c. potassium chloride present at 108 mg/gel capsule,
d. magnesium oxide present at 25 mg/gel capsule, and
e. D-Mannose present at 75 mg/gel capsule.

7. A method of treating dehydration comprising the step of: administering two of the gel capsules according to claim 6 to a dehydrated subject.

8. A method of preventing or reducing the incidence of dehydration associated with strenuous physical exercise comprising the step of: administering two of the gel capsules according to claim 6 to a human subject prior to or during strenuous physical exercise.

9. A method of preventing or reducing the incidence of dehydration associated with strenuous physical exercise in hot climates or work environments, comprising the step of: administering two of the gel capsules according to claim 6 to a human subject prior to or during strenuous physical exercise.
